(19) United States
(12) Patent Application Publication
(10) Pub. No.: US 2020/0245940 A1
(43) Pub. Date: Aug. 6, 2020

(54) A NEUROLOGICAL ELECTRODE SYSTEM FOR SENSITIVE SKIN

(71) Applicant: Rhythmlink International, LLC, Columbia, SC (US)

(72) Inventors: Harrison Floyd, Columbia, SC (US); Daniel McCoy, Columbia, SC (US)

(21) Appl. No.: 16/639,281

(22) PCT Filed: Aug. 17, 2018
(86) PCT No.: PCT/US18/00327

§ 371 Date: Feb. 14, 2020

Related U.S. Application Data
(60) Provisional application No. 62/546,678, filed on Aug. 17, 2017.

(51) Int. Cl. A61B 5/00; A61B 5/0478

(52) U.S. Cl. CPC A61B 5/6832; A61N 1/0496; A61B 5/0478; A61B 5/68335

(57) ABSTRACT

An electrode system for neurological monitoring is disclosed, including a housing with a recess dimensioned to hold an electrode. A conductive is applied to said electrode to limit direct contact by said electrode to a patient's skin. The recess defines a wall in said housing that engages the patient's skin. A thin bead of adhesive applied to the top surface of the wall secures the housing, and therefore the electrode, to the patient. Finer electrical conductor wires in a more flexible insulator lead from the electrode to a connector that fits into a neurological monitoring amplifier.

### TECHNOLOGICAL FIELD

The present disclosure relates generally to neurological electrodes.

### BACKGROUND

Neurological electrodes are used for monitoring and stimulating brain function. For example, when attached to the skin of the scalp of a patient, they can sense nerve impulses inside the brain. However, these electrodes may irritate the skin if they remain attached for a protracted time, and, eventually, cause the skin to breakdown, particularly where the stress of the electrode on the scalp is concentrated, for example, where the rim of a cup-type electrode contacts the skin.

For most patients and most types of neurological monitoring, the effect of the electrodes on the patient's skin is not a significant concern. In caring for newborn babies and others whose skin is sensitive, skin damage is of greater concern.

### SUMMARY

According to its major aspects and briefly recited, herein is disclosed an electrode system for neurological monitoring of patients with sensitive skin, such as newborn infants, the elderly, malnourished, and burn victims. The present electrode system includes an electrode, a housing, a lead wire, and a connector on the distal end of the wire. The electrode itself is made of a softer material than metal or epoxy, and the electrode is placed in electrical connection with the skin of the patient in a way to minimize physical connection, namely, via a conductive coating or gel between the electrode and the patient. The electrode and conductive gel are held within a recess in a foam housing. The housing is placed against the patient's head with the recessed side facing the head. A thin adhesive on the top surface of the wall that defines the recess and conductive gel is placed in the recess with the electrode. The housing thus both holds the electrode and conductive gel in the recess on the patient's side and cushions the patient's head against external forces. In addition, the electrode wires are thinner and more flexible, and they are attached to the electrode without welds or solder joints so as to minimize patient discomfort.

The softer conductive gel compared to the electrode extends the detection range of the electrode while limiting the electrode's direct contact with sensitive skin of the patient. The present electrode, which may be a “waffle-type” design that is more sensitive to electrical signals from the brain, is able to provide brain wave information with the intermediate presence of the conductive gel.

The present electrode is secured to the skin by a thin ring of film adhesive rather than a thicker adhesive “sticky pad” to limit skin damage on removal.

Those skilled in the use and design of neurological electrodes will appreciate the many advantages of the present electrode system from a careful reading of the following Detailed Description in view of the accompanying drawings.

### BRIEF DESCRIPTION OF THE DRAWINGS

In the figure,

FIG. 1 is an exploded view of the electrode system according to an aspect of the disclosure;

FIG. 2 is a side, cross-sectional, exploded view of a portion of the present electrode system, according to an aspect of the disclosure;

FIG. 3A is a side, cross-sectional view of a portion of the present electrode system prior to use, according to an aspect of the disclosure;

FIG. 3B is a side, cross-sectional view of a portion of the present electrode system in use, according to an aspect of the disclosure;

FIG. 4 is a top view of a set of electrode systems on a single release paper.

### DETAILED DESCRIPTION

The present neurological electrode system reduces patient discomfort and delays skin breakdown compared to other electrodes. In other respects, the present electrode system may be used in electro-encephalograph (EEG) or intra-operative monitoring (IOM) applications in the same manner as other neurological electrodes, that is, for stimulating a patient's brain or recording brain activity.

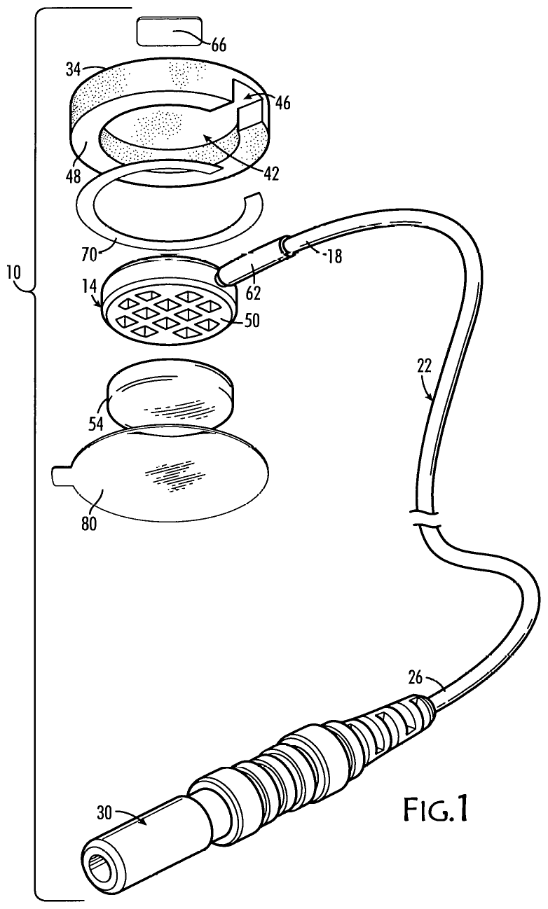

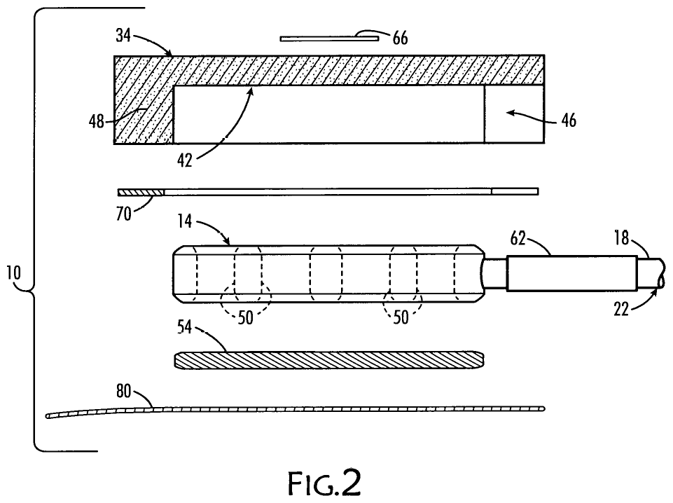

Referring now to FIGS. 1, 2 3A and 3B, the present electrode system 10 includes a housing 34 made of a resilient material such as a foamed plastic, for example, a closed-cell, cross-linked, nitrogen-impregnated, polyethylene foam. Housing 34 is therefore resilient and intended to serve as a cushion against external pressure such as when the head of the patient is resting against a pillow and the electrode is between the pillow and the patient's head. Housing 34 has a recess 42 formed therein and defined by a wall 48. The interior of recess 42 is dimensioned to receive an electrode 14, and may be dimensioned to hold electrode 14 securely.

Electrode 14 may be made of a flexible material such as silicone rubber or acrylonitrile butadiene styrene (ABS) with an electrically conductive coating such as mixture of silver and silver chloride, and may be impregnated with a conductive material such as copper fibers or carbon fibers, such as graphite fibers. Electrode 14 may be formed to have passages 50 in it, and which may be through passages 50 to provide electrode 14 with more contact with a conductive gel 54 than if electrode did not have passages 50. Electrode 14 may be, for example, the WEBB electrode manufactured and sold by Rhythmlink, International, Inc.

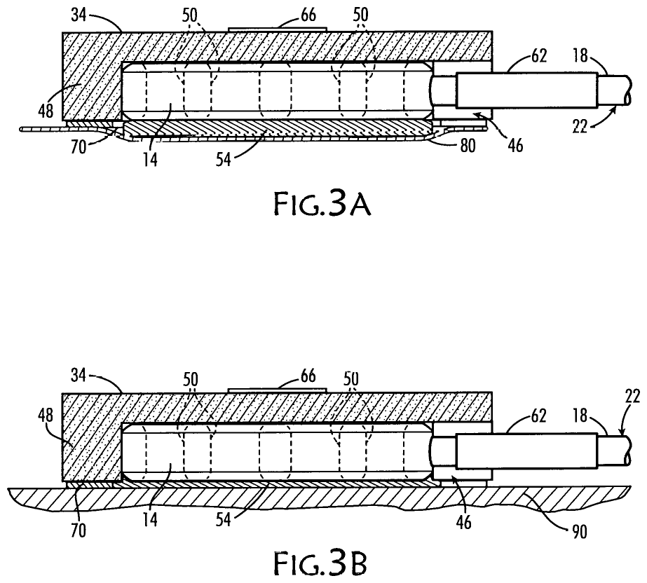

Passages 50 in electrode 14 that receive an in-flow of conductive gel 54 help improve inter-surface contact between conductive get 34 and electrode 14. A neonatal grade conductive gel may be used. Conductive gel 54, when in contact with electrode 14, extends the effective range of electrode 14 for detecting or sending electrical signals. Electrode 14 is not attached to the patient's skin 90 (best seen in FIG. 3B) and may not be in direct contact with the patient's skin 90. Electrode 14, however, can sense and transmit electrical signals so long as conductive gel 54 is in contact with skin 90 of the patient and gel 34 is in contact with electrode 14.

The surface 38 of wall 48 may include an adhesive ring 70 that will hold housing 34 to skin 90 of patient, which adhesive may be a thin laminate adhesive. Adhesive ring 70 may be applied to surface 38 of wall 48 as a thin ring. A neonatal grade of skin adhesive may be used such as a hydrogel-type adhesive to limit the extent and severity of skin injuries on removal.

Prior to use, a release paper 80 may be applied over wall 48 and recess 42 to protect adhesive ring 70, conductive gel 54, and electrode 14 prior to use. Release paper 46 is then peeled from electrode system 10 and the recess side of housing 34 with adhesive 70 is placed against the patient's skin 90.

The side of electrode 14 opposing the side with recess 42 may optionally carry an indicator of the position on the scalp of a patient designated for that electrode 14. The position designation may be selected from those position designations in a standard set of position designations for the human head, which position designations are familiar to those of ordinary skill in the art of neurological monitoring.

Electrical conductors 22 have a first end 18 connected to electrode 14 in a manner that avoids hard surfaces such as welds or solder joints. For example, first end 18 of an electrical conductor 22 may be inserted into a sleeve 62 attached to electrode 14 and pass through an opening 46 in wall 48. A distal end 26 of electrical conductors 22 terminate in a connector 30 that is selected to be received directly by a neurological monitoring amplifier (not shown). Electrical conductors 22 used herein may also be made of a thinner gauge of metal, such as 18 gauge or higher, or of carbon. Electrical conductors 22 may comprise a wire having an electrically insulating coating of a more flexible electrically insulating material, such as one made of complex polymers like perfluoroalkoxy.

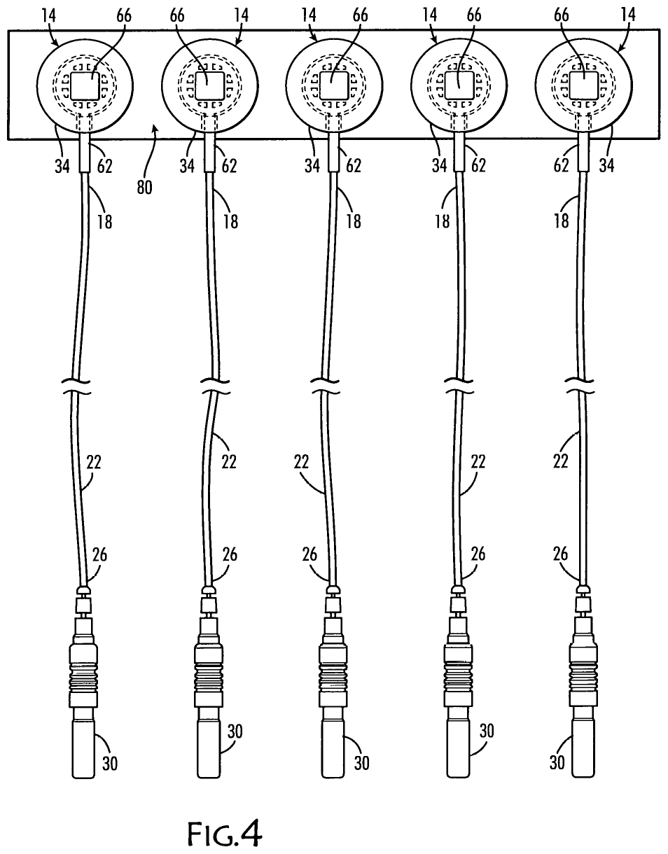

As seen in FIG. 4, a set of electrode systems 10 as described above may be needed for monitoring. Five electrode systems 10 are illustrated in FIG. 4, however, a different number may be required. A portion or all of the electrode systems 10 required for neurological monitoring or stimulating of an infant may be attached to a single, extended release paper 80 for convenience in a row and with the side of housing 34 opposite recess 42 facing down so that a position indicator 62 may be facing up toward the technician assigned to apply electrode systems 10.

Those skilled in the art of neurological electrodes will appreciate that many modifications and substitutions may be made in the electrode system as herein described without departing from the spirit and scope of the present disclosure.

What is claimed is:

1. An electrode system, comprising:
(a) a housing with wall defining a recess formed in said housing;
(b) an electrode in said recess;
(c) a conductive gel in said recess and in contact with said electrode;
(d) an electrical conductor having a first end and a second end, said first end of said electrical conductor being connected to said electrode in said recess; and
(e) a connector connected to said second end of said electrode wherein said electrode is made of silicone.

2. The electrode system of claim 1, wherein an opening is formed in said wall for said electrical conductor to pass therethrough.

3. The electrode system of claim 1, wherein said electrode system further comprises
(a) an adhesive on said wall; and
(b) a release paper on said adhesive.

4. The electrode system of claim 1, wherein said housing is made of foamed plastic.

5. The electrode system of claim 1, wherein said electrode has passages formed therein, and wherein said conductive gel penetrates into said passages when said electrode and said conductive gel are in said recess.

6. The electrode system of claim 1, wherein said electrical conductor is made of carbon.

7. The electrode system of claim 1, wherein said electrode is made of silicone rubber.

8. The electrode system of claim 1, wherein said electrode is made of silicone rubber filled with conducting fibers.

9. The electrode system of claim 1, wherein said electrode is made of acrylonitrile butadiene styrene.

10. The electrode system of claim 1, wherein said electrode is made of acrylonitrile butadiene styrene with a conductive coating.

11. The electrode system of claim 1, wherein said electrode is made of acrylonitrile butadiene styrene with a conductive coating made of a mixture of silver and silver-chloride.

12. The electrode system of claim 1, further comprising a sleeve, and wherein sleeve is operable to connect said electrical conductor to said electrode into said sleeve.

13. The electrode system of claim 1, wherein said housing is made of polyethylene foam.

14. The electrode system of claim 1, wherein said conductive gel is a neonatal conductive gel.

15. The electrode system of claim 1, wherein said housing carries an electrode position designation.
